(19) United States
(12) Patent Application Publication
(10) Pub. No.: US 2013/0211422 A1
(43) Pub. Date: Aug. 15, 2013

(54) COMPACT ROTARY ACTUATOR WITH INTERNAL PLANETARY

(71) Applicant: Intuitive Surgical Operations, Inc., (US)

(72) Inventor: David W. Bailey, Portola Valley, CA (US)

(73) Assignee: INTUITIVE SURGICAL OPERATIONS, INC., Sunnyvale, CA (US)

(21) Appl. No.: 13/767,801

(22) Filed: Feb. 14, 2013

Related U.S. Application Data
(60) Provisional application No. 61/599,227, filed on Feb. 15, 2012.

(51) Int. Cl. A61B 19/00

(52) U.S. Cl. CPC A61B 19/2203
USPC 606/130

(57) ABSTRACT

A rotary actuator for use in surgical instruments is provided, including a plurality of engine elements placed symmetrically around a rotation axis; a disk in contact with the engine elements, centered on the rotation axis, the engine elements providing a first rotation to the disk; an input shaft coupled to the disk to rotate with the first rotation; and a coupling mechanism to provide a second rotation to an output shaft from the first rotation of the input shaft. A surgical manipulator is also provided, including a base to provide stability and support; a first rotary actuator as above coupled to the base; and a first arm coupled to the rotary actuator.

### RELATED APPLICATIONS

This application claims priority to U.S. Provisional Application Ser. No. 61/599,227, filed on Feb. 15, 2012, which is herein incorporated by reference in its entirety.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

1. Field of the Invention

Embodiments described herein relate to actuators that may be utilized in the field of surgical instruments, and more particularly to the field of robotically actuated surgical instruments.

2. Description of Related Art

In the field of robotic surgery and machine-aided surgical procedures rotary actuators are commonly used to manipulate probes and other surgical instruments. Rotation of components about more than one degree of freedom often makes use of more than one rotary actuator acting about a specific rotation axis. Remotely controlled surgical instruments typically build up a heavy structure having elongated arms and components. Rotary actuators associated with such mechanisms should be able to produce a high torque in order to produce a smooth, repetitive and accurate motion. State-of-the-art motors and actuators are not adapted for high torque delivery, unless complicated mechanical adjustments are made. In some cases, specialized motors are used for specific components that require a high torque output, making the overall instrument design complex and expensive.

Therefore, there is a need for rotational mechanisms that provide a high torque and a high degree of angular motion accuracy in a compact device. Furthermore, rotational mechanisms simply adjustable to different uses are desirable.

### SUMMARY

According to embodiments disclosed herein a rotary actuator for use in surgical instruments may include a plurality of engine elements placed symmetrically around a rotation axis; a disk in contact with the engine elements, centered on the rotation axis, the engine elements providing a first rotation to the disk; an input shaft coupled to the disk to rotate with the first rotation; and a coupling mechanism to provide a second rotation to an output shaft from the first rotation of the input shaft.

According to embodiments disclosed herein a surgical manipulator may include a base to provide stability and support; a first rotary actuator coupled to the base; and a first arm coupled to the rotary actuator. In some embodiments the first rotary actuator may include a plurality of engine elements placed symmetrically around a first rotation axis; a disk in contact with the engine elements, centered on the rotation axis, the engine elements providing a first rotation to the disk; an input shaft coupled to the disk to rotate with the first rotation; and a coupling mechanism to provide a second rotation to an output shaft from the first rotation of the input shaft.

These and other embodiments of the present invention will be described in further detail below with reference to the following drawings.

### BRIEF DESCRIPTION OF THE DRAWINGS

FIG. 1 illustrates a torque/speed chart for a rotary actuator according to embodiments disclosed herein.

FIG. 2A illustrates a sagittal view of a rotary actuator according to embodiments disclosed herein.

FIG. 2B illustrates a cross-sectional view of a rotary actuator according to embodiments disclosed herein.

FIG. 2C illustrates a sagittal view of a rotary actuator according to embodiments disclosed herein.

FIG. 3 illustrates a cross-sectional view of a coupling mechanism for use in a rotary actuator according to embodiments disclosed herein.

FIG. 4 illustrates a sagittal view of a rotary actuator according to embodiments disclosed herein.

FIG. 5 illustrates a cross-sectional view of a rotary actuator according to embodiments disclosed herein.

FIG. 6 illustrates a cross-sectional view of a coupling mechanism for use in a rotary actuator according to embodiments disclosed herein.

FIG. 7 illustrates a surgical manipulator including rotary actuators according to embodiments disclosed herein.

In the figures, elements having the same reference number have the same or similar functions.

### DETAILED DESCRIPTION

Embodiments disclosed herein include a compact rotary actuator used for a surgical instrument. According to some embodiments, the surgical instrument may be remotely controlled, for example in a robotic application. Further according to some embodiments, the use of the surgical instrument may determine the materials used and dimensions of a rotary actuator. For example, in some embodiments a rotary actuator may be employed in applications involving a Magnetic Resonance Imaging (MRI) procedure. In such cases, the use of non-metallic or non-ferromagnetic components may be desirable, to avoid interference with the magnetic field of the MRI instrument. An interference with the magnetic field of the MRI instrument may distort or destroy the image produced. Also, metallic or ferromagnetic components may be a safety hazard for the patient and or personnel in case the component becomes ballistic in the magnetic field of the MRI instrument.

Also, in embodiments for use with surgical instruments in an MRI environment spatial constrains may preclude access to a surgical point in the patient from certain directions. Thus, a surgical instrument having elongated form factors may be desirable in MRI applications. A rotary actuator providing pitch and yaw degrees of freedom in such embodiments may deliver a high torque in order to provide accurate and precise motion of heavy surgical components.

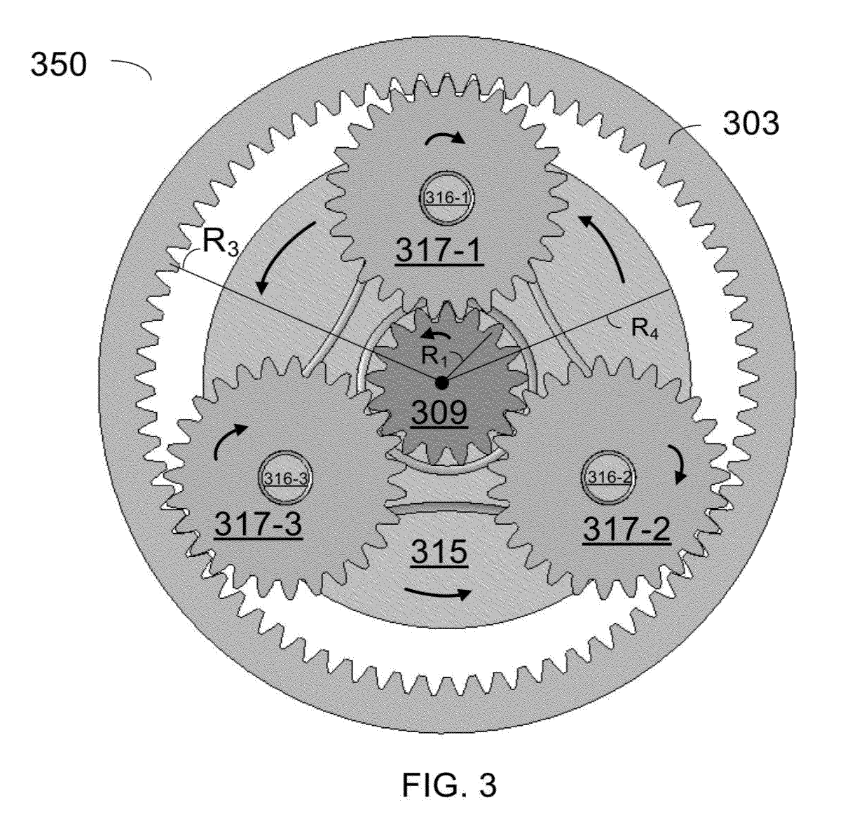

According to embodiments such as disclosed herein, a rotary actuator may include a coupling mechanism that increases the output torque provided by an engine element in the rotary actuator. In some embodiments, the coupling mechanism may be a planetary gear system, such as described in detail in FIG. 3. A planetary gear system includes an input gear that may be at the center, forming a ‘sun’ element. Planet gears may be coupled to rotate around the input gear, tracking along an exterior gear that may be fixed. The planet gears may be attached to an output shaft. Thus, a planetary system provides an input shaft having typically smaller diameter, higher rotational speed, and lower torque relative to an output shaft.

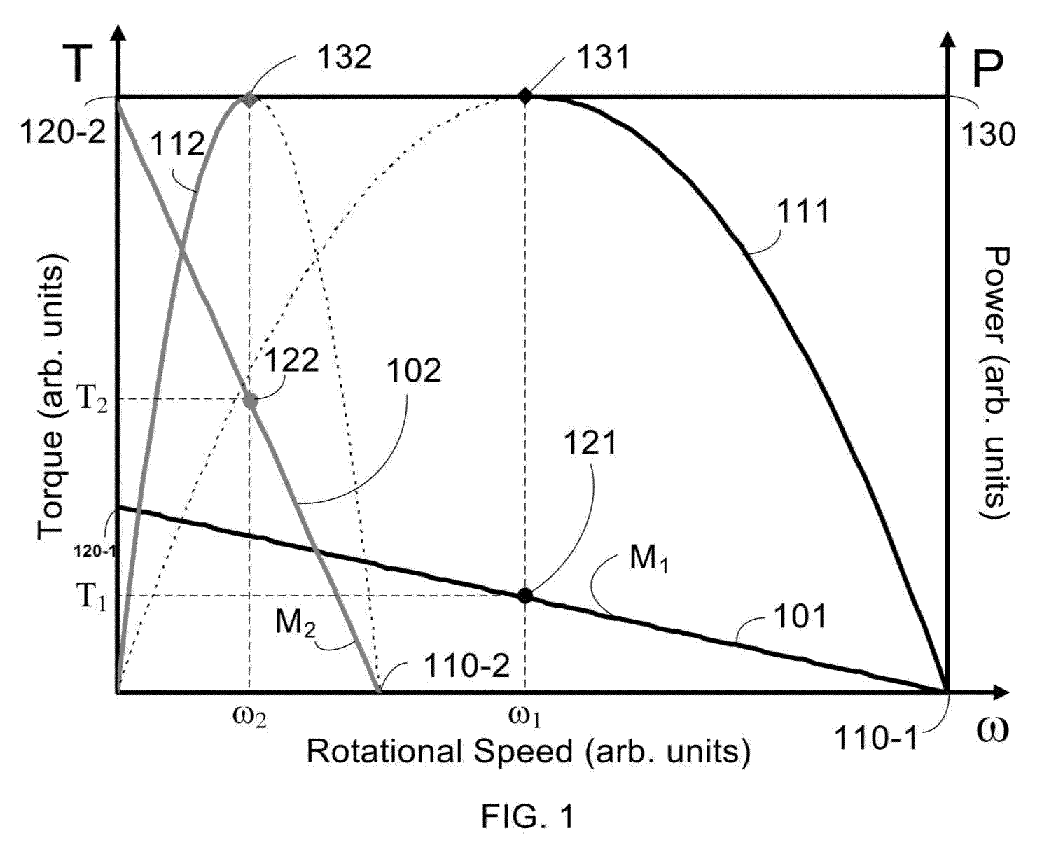

FIG. 1 illustrates a torque/speed chart 100 for a rotary actuator according to some embodiments disclosed herein. FIG. 1 includes torque/speed curves 101 and 102. A torque/speed curve is a plot of the torque provided by a rotary actuator for a given rotational speed, according to embodiments disclosed herein. Chart 100 includes a torque value (T) in the ordinate axis and a rotational speed (ω) value in the abscissa axis. Chart 100 also shows a secondary ordinate axis for the power, P, delivered by a rotary actuator according to some embodiments. The power P may be given by the expression:

P=T·ω (1)

A rotary actuator according to some embodiments disclosed herein may have a characteristic torque/speed curve 101 or 102. In some embodiments, curve 101 having slope M₁ forms a line from a maximum torque 120-1 provided at zero speed, to zero torque provided at a maximum speed 110-1. In some embodiments, curve 102 having slope M₂ forms a line from a maximum torque 120-2 provided at zero speed, to zero torque provided at a maximum speed 110-2. The slope M of a torque/speed curve as in chart 100 indicates the torque delivered by a rotary actuator as in embodiments disclosed herein for a given rotational speed. Curve 111 shows the power delivered by a rotary actuator consistent with curve 101. Curve 112 shows the power delivered by a rotary actuator consistent with curve 102. Note that the maximum power 130 (Pₘₐₓ) delivered by a rotary actuator is the same for embodiments consistent with curve 101 and embodiments consistent with curve 102. More generally, the value of Pₘₐₓ 130 is determined by the engine used to provide motion in a rotary actuator according to embodiments disclosed herein. A rotary actuator may provide a torque and a rotational speed according to curve 101 or curve 102, depending on the details of the mechanical coupling in the actuator. For example, dimensions of the mechanical elements may be adjusted to choose the value of slope M. Also, gear systems may be included to adjust the slope M of a torque/speed curve as shown in chart 100.

For example, having |M₁|<|M₂| may indicate that a rotary actuator consistent with curve 102 delivers a higher level of power at lower speeds, compared to a rotary actuator consistent with curve 101 for a given rotational speed (cf. FIG. 1). As the rotational speed increases, a configuration as in curve 101 having slope M₁ may deliver a higher power. Furthermore, in embodiments disclosed herein |M₁|<|M₂| may indicate that maximum speed 110-1 is larger than maximum speed 110-2 (cf. FIG. 1). Also according to embodiments consistent with the present disclosure |M₁<|M₂| may indicate that maximum torque 120-2 is larger than maximum torque 120-1 (cf. FIG. 1). FIG. 1 illustrates a rotary actuator consistent with curve 101 having T and ω given by point 121. The power delivered by the rotary actuator in such configuration is illustrated by point 131. By adjusting the mechanical coupling of the same actuator according to embodiments disclosed herein a configuration consistent with curve 102 may have T and ω as in point 122. The power delivered by the rotary actuator is given by point 132. According to embodiments disclosed herein, power value 132 in configuration 122 is the same as power value 131 in configuration 121. Further according to embodiments consistent with the present disclosure, the configuration of rotary operator in curves 101 and 102 according to points 121 and 122 may correspond to a maximum power Pₘₐₓ 130 delivered by a rotary actuator. Thus, by adjusting a mechanical coupling in a rotary actuator from configuration 121 to configuration 122 a higher T is delivered, by using the same power in the actuator.

While FIG. 1 depicts torque T and rotational speed ω (cycles/sec), it should be understood that a similar curve involving force F (in Newtons) and linear speed v (in mm/s) may be used in embodiments where engine elements operate in a linear fashion. The conversion from linear variables to rotational variables is performed through a radius R, of an element upon which a linear component may operate. In some embodiments, a rotary actuator with a shaft having radius R (mm) may provide a maximum torque of 4×R Newtons·mm (N·mm) and a maximum rotational speed of 1/(2·πR)×250 rad/s. In some embodiments, the radius R may be about one (1) inch, or approximately 25 mm.

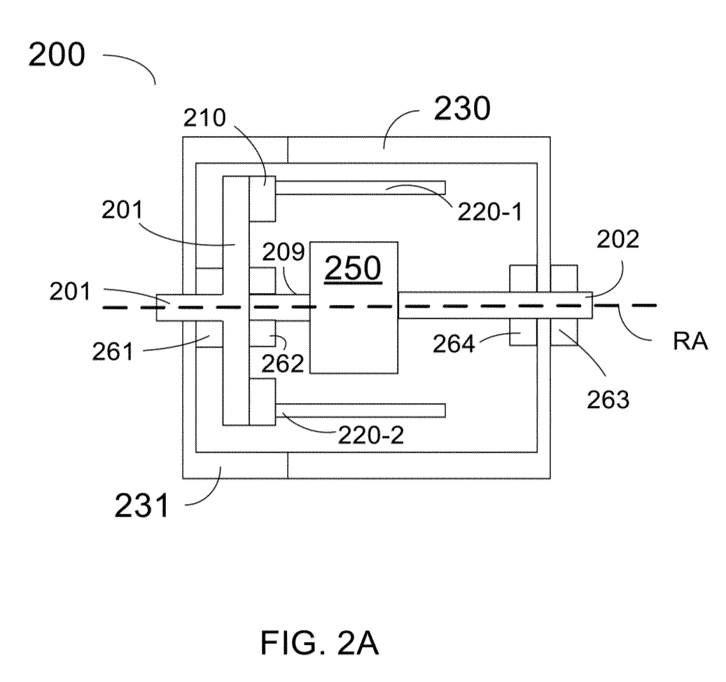

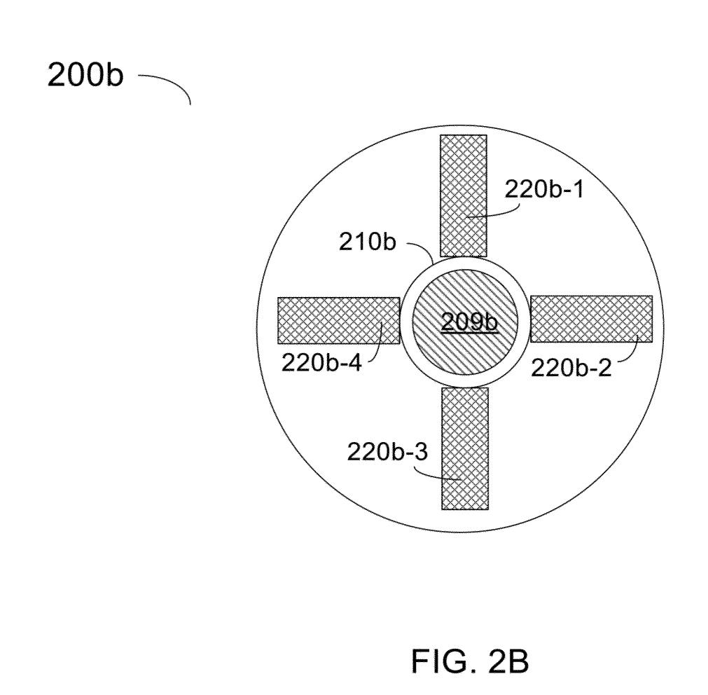

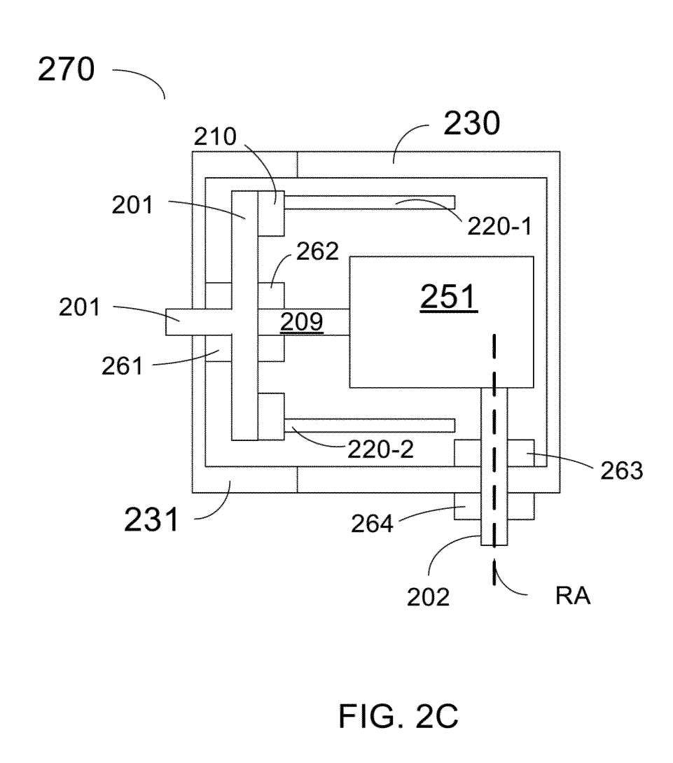

FIG. 2A illustrates a sagittal view of a rotary actuator 200 according to embodiments disclosed herein. Rotary actuator 200 includes engine elements 220 as part of the engine used to provide motion to a disk 210 (elements 220-1 and 220-2 are shown). There may be any number of engine elements 220. Some embodiments may have engine elements not included in the sagittal plane of FIG. 2A. Rotary actuator 200 may include any number of engine elements 220 as feasible in terms of size and power of the engine mechanism. According to embodiments consistent with the present disclosure, elements 220 may be piezo-electric actuators having a longitudinal axis of operation. In some embodiments, elements 220-1 and 220-2 are arranged circumferentially about the rotary actuator axis RA (in the sagittal plane of FIG. 2A). Engine elements 220 may be cylindrical or elongated, with their longitudinal axis of operation aligned to the RA. Piezo-electric actuators such as those included in elements 220-1 and 220-2 may be desirable in applications involving surgical instruments used in an MRI environment. Piezo-electric materials are non-ferromagnetic, thus their interaction with the magnetic field in an MRI environment is negligible. In embodiments consistent with the present disclosure, piezo-electric elements 220 are configured to bend along their longitudinal axis of operation, making an elliptical pattern with the end of the element in direct contact with disk 210. By precisely adjusting the periodic bending of piezo-electric element 220, a clock-wise or counter-clockwise rotation may be provided to disk 210.

Engine elements 220-1 and 220-2 provide a rotational motion to disk 210 about its axis, in the plane of the figure (sagittal plane of rotary actuator 200). Disk 210 may be formed of a ceramic material, according to some embodiments. Disk 210 is coupled to input shaft 201. A bearing 261 allows input shaft 201 to rotate freely while being securely held within an actuator frame 230. Rotary actuator 200 may also include a bearing 262 to reduce friction for the rotation of disk 210. According to embodiments consistent with the present disclosure, rotary actuator 200 may include a coupling mechanism 250. Coupling mechanism 250 provides a rotational motion to output shaft 202 from the rotational motion of input shaft 201 through coupling input 209. In embodiments consistent with the present disclosure, coupling mechanism 250 may include a gear train having an input axis and an output axis being co-axial with the RA. In embodiments consistent with the present disclosure, coupling mechanism 250 may be contained within cap 231, attached to actuator frame 230. Bearings 263 and 264 allow output shaft 202 to rotate freely while being securely held within frame 230. According to embodiments consistent with the present disclosure any number of bearings such as bearings 261, 262, 263 and 264 may be included in different portions of shafts 201 and 202, and in coupling mechanism 250. According to some embodiments, input shafts 201 and 202, and coupling input 209 have an axis aligned with the RA.

FIG. 2B illustrates a cross-sectional view of a rotary actuator 200b according to embodiments disclosed herein. In actuator 200b engine elements 220b may be radially mounted around disk 210b. For example, engine elements 220b-1, 220b-2, 220b-3, and 220b-4 may have their elongated axis oriented in the plane of disk 210b. In such configuration, engine elements 220b act radially on an edge of disk 210b to produce a rotation. According to embodiments consistent with the present disclosure other components in actuator 200b may be as in actuator 200, such as coupling input 209b (cf. FIG. 2A).

In some embodiments, different components in rotary actuator 200 may be formed of a ceramic material, either by machining the pieces or by molding. Furthermore, some components may be machined or molded together as a single piece. In some embodiments, disk 210 and input shaft 201 may be machined or molded together as a single piece. Further according to some embodiments of the present disclosure, the axes of input shaft 201 and output shaft 202 may form an angle relative to each other. This is described in detail in relation to FIG. 2C, below.

FIG. 2C illustrates a sagittal view of a rotary actuator 270 according to embodiments disclosed herein. In some embodiments, the RA is associated to the axis of output shaft 202, which may form a perpendicular angle with the axis of input shaft 201. In such configuration, coupling mechanism 251 may include a gear system adapted to change the orientation from an input axis to an output axis. All other components shown in FIG. 2B may be as described in detail above in relation to FIG. 2A. Some embodiments consistent with the disclosure herein may include an RA forming an angle different from zero (0) and not necessarily perpendicular with respect to the axis of input shaft 201.

In some embodiments consistent with the present disclosure, input shaft 201 may have a rotational motion consistent with curve 101 (cf. FIG. 1), having slope M₁. Further according to some embodiments, output shaft 202 may have a rotational motion consistent with curve 102 (cf. FIG. 1), having slope M₂. Thus, in some embodiments coupling mechanism 250 enables the switch from a rotational configuration as in point 121 to a rotational configuration as in point 122 (cf. FIG. 1). Note that in such embodiments the output power of rotational actuator 200 may be as in point 132 and substantially equal to the input power in point 131 (cf. FIG. 1). In some embodiments, coupling mechanism 250 may dissipate an amount of input energy, and thus output power 132 may be somewhat lower than input power 131. It is desirable that coupling mechanism 250 dissipate as little energy as possible, while switching the rotational configuration from point 121 to point 122.

In embodiments consistent with the present disclosure coupling mechanism 250 provides a trade-off between T and ω from input shaft 201 to output shaft 202. In some embodiments as disclosed above coupling mechanism 250 couples input configuration 121 to output configuration 122 (cf. FIG. 1). In such embodiments, a low input T is converted to a high output T, and a high input ω is converted to a low output ω. Such an embodiment may be highly desirable for a rotary actuator used in a surgical instrument within an MRI environment, due to the availability of a high torque output.

According to some embodiments, rotary actuator 200 may be adapted to the force capabilities of an individual engine element 220. In such embodiments a determination of torque-speed (and therefore power) desired for the actuator output 202 is made based upon the application intended. From the peak power 130 desired by the actuator (cf. FIG. 1), the number of engine elements 220 is determined. Engine elements 220 are arranged into a circle next to disk 210 determining the operating radius and torque T₁ of the input shaft. Therefore, a torque-speed curve 101 is established for the input shaft (cf. FIG. 1). Coupling mechanism 250 includes a gear train to convert from input shaft curve (101) to output curve (102) according to a desired output torque T₂. According to some embodiments, the desired output torque may determine a gear ratio of a single stage that is large, such as 22:1. In some embodiments, such a large gear ratio may not fit inside a constrained actuator frame 230 in a single stage. In such configurations it may be desirable to use a gear train including more than one gear stage so that the torque correction factor is multiplied to produce a high output torque T₂. In some embodiments, coupling mechanism 250 is determined by the geometry of a specific application. In such cases, the number of engine elements 220 may be increased to produce an output torque T₂ as high as desired. Thus, for a given torque ratio provided by a coupling mechanism 250 limited by geometric constraints, the output torque T₂ may be increased by increasing the input torque T₁, adding engine elements 220.

In some embodiments, actuator 200 as illustrated in FIGS. 2A and 2B can be a compact actuator. As discussed above, a compact actuator can be formed by first determining the torque speed curve that fulfills a particular task. A determination of the torque-speed desired for the actuator output 202 can be made based on the intended application. From the peak power 130 requested of actuator 200 (cf. FIG. 1), the number of engine elements 220 can be determined. The number of engine elements 220 is the number of individual elements that will supply the peak power 130 to perform the intended application. The compact actuator can be formed by arranging the number of engine elements 220 in as small a circle as practical. The coupling mechanism 250 can then be determined so as to shift the torque speed of the tightly packed motor elements 220 in actuator 200 to the performance to perform the intended application.

A detailed description of a coupling mechanism, such as mechanism 250 used in actuator 200 shown in FIG. 2A, will be provided in relation to FIG. 3, below.

FIG. 3 illustrates a cross-sectional view of a coupling mechanism 350 for use in a rotary actuator according to embodiments disclosed herein. Coupling mechanism 350 may include a planetary gear system. Coupling mechanism 350 includes coupling input 309 having a ‘sun’ gear at the center with a radius R₁, providing a rotational motion to ‘planet’ gears 317. In FIG. 3, three planet gears 317-1 through 317-3 are illustrated. The number of planet gears 317 used in coupling mechanism 350 is non-limiting. In some embodiments, four planet gears 317 may be used, or more. It is desirable that planet gears 317 be placed around the sun gear, symmetrically about the axis of the sun gear in coupling input 309. This may provide mechanical stability and balance, thus reducing energy dissipation in coupling mechanism 350. Planet gears 317 include shafts 316 on their rotation axes. According to some embodiments, shafts 316-1 through 316-3 may be coupled to a disk 315. Coupling mechanism 350 includes exterior gear 303 having radius R₃, providing a track for planet gears 317. Embodiments of coupling mechanism 350 are not limiting by the illustration in FIG. 3. Some embodiments of coupling mechanism 350 may produce an increase in rotational speed from input shaft 201 to output shaft 202. In such configuration the planetary gear system would be different from what is illustrated in FIG. 3.

In embodiments consistent with the present disclosure, coupling input 309 may be as coupling input 209 in rotary actuator 200 (cf. FIGS. 2A and 2B). In some embodiments disk 315 may be coupled to an output shaft such as shaft 202 in rotary actuator 200 (cf. FIGS. 2A and 2B). Exterior gear 303 may be fixed to actuator frame 230 in coupling mechanism 250 (cf. FIG. 2A). Thus, in embodiments consistent with the present disclosure shaft 309 may provide a torque T₁ and rotational speed ω₁. The motion of planet gears 317 provides a torque T₃ and rotational speed ω₃ to disk 315. Exterior gear 303 remains stationary in the plane of FIG. 3, according to some embodiments.

FIG. 3 illustrates the sun gear in coupling input 309 rotating counter-clockwise, planet gears 317 rotating clock-wise, and disk 315 rotating counter-clockwise. It is understood that the direction of rotation of each of the gears in the planetary system of FIG. 3 may be reversed without loss of generality. Embodiments having the opposite rotation direction in the sun gear of coupling input 309, planet gears 317, and disk 315, are also consistent with the present disclosure.

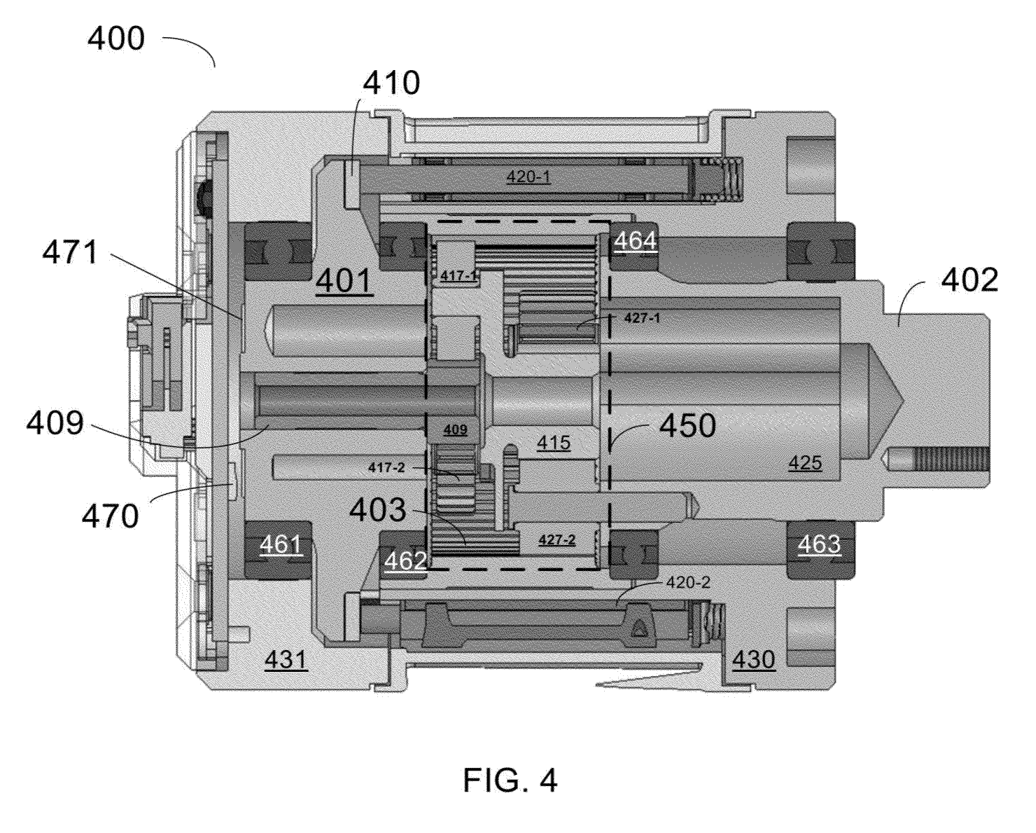

Thus, in some embodiments an input rotation having a torque/speed configuration including point (T₁, ω₁) is coupled to an output rotation having a torque/speed configuration including point (T₃, ω₃), with T₁<T₃ and ω₃<ω₁. The design of coupling mechanism 350 may adjust the values of R₁, and R₂ in order to obtain the desired output T₃, according to the application. In some embodiments, in addition to adjusting the relative sizes of gears as in coupling mechanism 350, a coupling mechanism may include a plurality of planetary stages such as mechanism 350. This allows for further adjustments to the output torque and speed of a rotary actuator consistent with the present disclosure. This is described in detail below in relation to FIG. 4. In some embodiments, to provide strength to coupling mechanism 350, the teeth on the sun gear in coupling input 309 may be wider than the teeth on planet gears 317. According to such embodiments the number of teeth in planet gears 317 may be lower than the radius ratio to the sun gear would determine. In such embodiments the torque ratio is not affected because the torque ratio is determined by the radius ratio.

FIG. 4 illustrates a sagittal view of a rotary actuator 400 according to embodiments disclosed herein. Rotary actuator 400 may include engine portions 420-1 and 420-2, frame 430, coupling input 409, disk 410, input shaft 401, output shaft 402 and bearings 461, 462, 463 and 464. In some embodiments, engine elements 420-1 and 420-2 may be as engine elements 220-1 and 220-2 described in detail above (cf. FIGS. 2A and 2B). Also according to some embodiments frame 430 may be as frame 230, disk 410 may be as disk 210, coupling input 409 may be as coupling input 209, input shaft 401 may be as input shaft 201, and output shaft 402 may be as output shaft 202 described in detail above (cf. FIG. 2A). In embodiments consistent with the present disclosure bearings 461, 462, 463, and 464 may be as bearings 261, 263, and 264 (cf. FIGS. 2A and 2B). Frame 430 is the component in rotary actuator 400 that holds the elements together, including the gear train in a couple mechanism, 450 according to some embodiments, and output shaft 402. In some embodiments, rotary actuator 400 may include a cap 431 holding input shaft 401.

Embodiments consistent with the present disclosure may include materials chosen for MRI safety, as discussed above. Embodiments consistent with the present disclosure may include components made of MRI-compatible materials. MRI-compatible materials typically have no ferrous metal components, according to some embodiments. In some embodiments, MRI-compatible materials may include non-ferrous metals that are not influenced by a magnetic field. For example, some embodiments include gears 403, 417, and 427 made out of non-ferromagnetic steel. In embodiments consistent with the present disclosure, rotary actuator 400 may include components formed from radiotranslucent materials. For example, a radiotranslucent material may have no metal component. In embodiments of the present disclosure used for X-ray scanning procedures, materials chosen for the components in rotary actuator 400 may be radio-opaque. Radio-opaque materials are opaque to X-ray radiation, so that the components made of these materials are easily visible in an X-ray machine. This allows the operator to proceed with caution as it manipulates a surgical instrument inside a patient.

Some embodiments include components shown in FIG. 4 made from materials such as aluminum for frame 430, cap 431 and shafts 401, 402. For example, some embodiments may use 6061-T6 aluminum for elements 430, 431, 401, 402, 403, and 409. Some embodiments include non-ferromagnetic stainless steels for gears 403, 417, 427, coupling input 409, and element 415. Some embodiments include bearings 461, 462, 463, and 464 made of ceramic materials such as silicon nitride.

Some embodiments of a rotary actuator as disclosed herein may include design considerations for components enabling heat dissipation. Some embodiments include a heat sink in rotary actuator 400. A heat sink may be a component having a mass and a large heat capacity placed in close proximity or in direct contact with the components in rotary actuator 400. For example, the heat sink may be in direct contact with or in close proximity to actuator frame 430. In some embodiments, a design consideration for choosing the mounting, arrangement, and materials in the components of a rotary actuator as rotary actuator 400 is the balance and stiffness of the assembly. Thus, for example, it may be desirable to arrange engine elements 420 symmetrically around the RA, in a plane perpendicular of the sagittal view in FIG. 4. Furthermore, gear components in coupling mechanism 450 may also be arranged symmetrically about the RA of rotary actuator 400. Symmetric arrangements may reduce the energy dissipation of coupling mechanism 450 in transferring from an input to an output rotational configuration (cf. points 131 and 132 in FIG. 1).

In embodiments consistent with the present disclosure rotary actuator 400 may include an encoder 470 for detecting the angular position of disk 410. According to some embodiments, encoder 470 may include an optical mechanism having a light source and a detector. In some embodiments the light source and the detector may be attached to cap 431, and a reflective element 471 may be attached to coupling input 409. Reflective element 471 may include evenly spaced marks so that the detector produces a modulated signal as input 409 rotates by the action of engine elements 420 on disk 410. A detection mechanism includes a signal processing circuit to count the number of marks passing in front of encoder 470 as disk 410 rotates. The precise angular position of input shaft 401 may be determined according to some embodiments. Thus, knowledge of the angular position of output shaft 402 may be deduced from the geometry of rotary actuator 400.

In some embodiments encoder 470 may be a motion sensor using a capacitance mechanism, or an electric mechanism for sensing position. Some embodiments may include an encoder 470-1 to measure the angular position of input shaft 401, and an encoder 470-2 to measure the angular position of output shaft 402. Further according to some embodiments the signal processing circuit to determine the position of input shaft 401 and output shaft 402 may be integrated to a motor controller. The motor controller may include a circuit providing power to engine elements 420, thus regulating the speed and torque of the rotational motion provided to input shaft 401. In some embodiments consistent with the present disclosure, encoder 470 is a reflectively coupled optical encoder.

Rotary actuator 400 may include coupling mechanism 450 to provide a rotation to output shaft 402 from a rotation in input shaft 401. As illustrated in FIG. 4, coupling mechanism 450 includes two planetary gear stages. Each planetary gear stage in coupling mechanism 450 may be as the planetary gear system described in detail above in relation to coupling mechanism 350. Thus, coupling input 409 may include the sun gear in a first stage of coupling mechanism 450. Gears 417-1 and 417-2 may be planet gears in the first stage. A third planet gear 417-3 (not shown) is not included in the sagittal plane of FIG. 4. As in coupling mechanism 350, the number of planetary gears used in the first stage of coupling mechanism 450 is not limiting. Planet gears 417 are rotationally coupled to disk 415, providing rotational motion as described in relation to disk 315 in coupling mechanism 350 (cf. FIG. 3). As disk 415 rotates, it provides input rotational motion for a second planetary stage in coupling mechanism 450. The second planetary stage has disk 415 as the sun gear, and planet gears 427 attached to output disk 425. The number of planet gears 427 in the second planetary stage is not limiting. In some embodiments of coupling mechanism 450 the number of planet gears 427 may be different form the number of planet gears 417 in the first stage. FIG. 4 illustrates planet gears 427-1 and 427-2, planet gear 427-3 (not shown) is not included in the sagittal plane of FIG. 4.

Coupling mechanism 450 includes exterior gear 403. According to some embodiments, a single exterior gear 403 may be used for the first and second planetary stages. Furthermore, some embodiments consistent with the present disclosure may include more than two planetary stages, all stages having a common exterior gear 403. According to embodiments disclosed herein, exterior gear 403 may be stationary relative to frame 430. Use of two or more planetary stages provides a broader range of adjustment for the output torque of a rotary actuator consistent with embodiments of the present disclosure. For example, if a single planetary system such as mechanism 350 produces an input/output torque factor of, say 2.0, then two such systems arranged in series as in mechanism 450 may provide an input/output torque factor of 4.0×4.0=16.0. Such an arrangement enhances the output torque, while maintaining the diameter of the planetary gear system within the same exterior gear 403. In some embodiments, the torque ratio provided by a first stage may be different form the torque ratio provided by a second stage. The specific value of the torque ratio provided by a first gear stage and a second gear stage depends on the application of the rotary actuator. Values of torque ratios provided herein are illustrative only and not limiting.

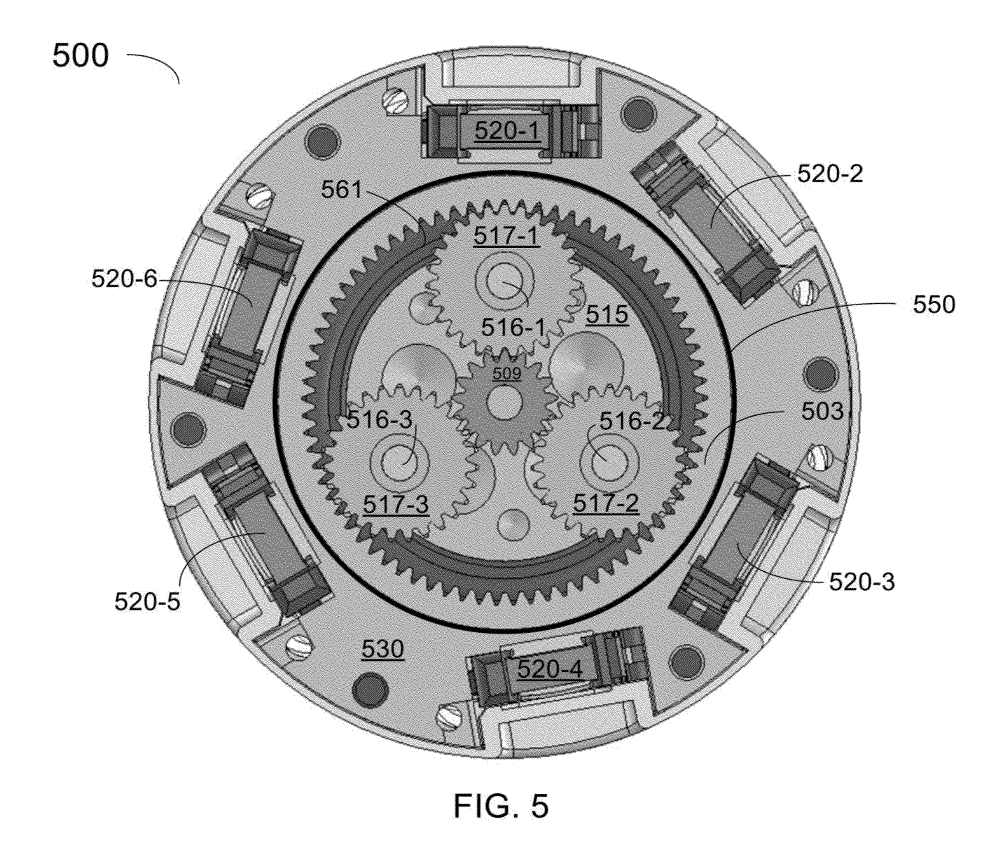

FIG. 5 illustrates a cross-sectional view of a rotary actuator 500 according to embodiments disclosed herein. Rotary actuator 500 includes engine elements 520-1 through 520-6 and coupling mechanism 550. According to some embodiments consistent with the present disclosure engine elements 520 may be piezo-electric actuators such as engine elements 220 described in detail above (cf. FIGS. 2A and 2B).

Coupling mechanism 550 may include a planetary gear stage having a sun gear in coupling input 509. The planetary gear stage in coupling mechanism 550 may also include planet gears 517-1 through 517-3, moving around the sun gear in coupling input 509, on the inner side of exterior gear 503. Exterior gear 503 may be stationary with respect to frame 530. Planet gears 517-1 through 517-3 have shafts 516 in their axes. Shafts 516-1 through 516-3 are coupled to disk 515 that may be attached to an output shaft (not included in the cross-sectional plane of FIG. 5). In some embodiments, the planetary system shown in FIG. 5 may be but one of a plurality of stages. Coupling mechanism 550 may include more than one stage having a planetary gear system as illustrated in FIG. 5. In such case, disk 515 may provide an input rotation to a second planetary stage further along the axis of rotary actuator 500 (out of the cross-sectional plane in FIG. 5).

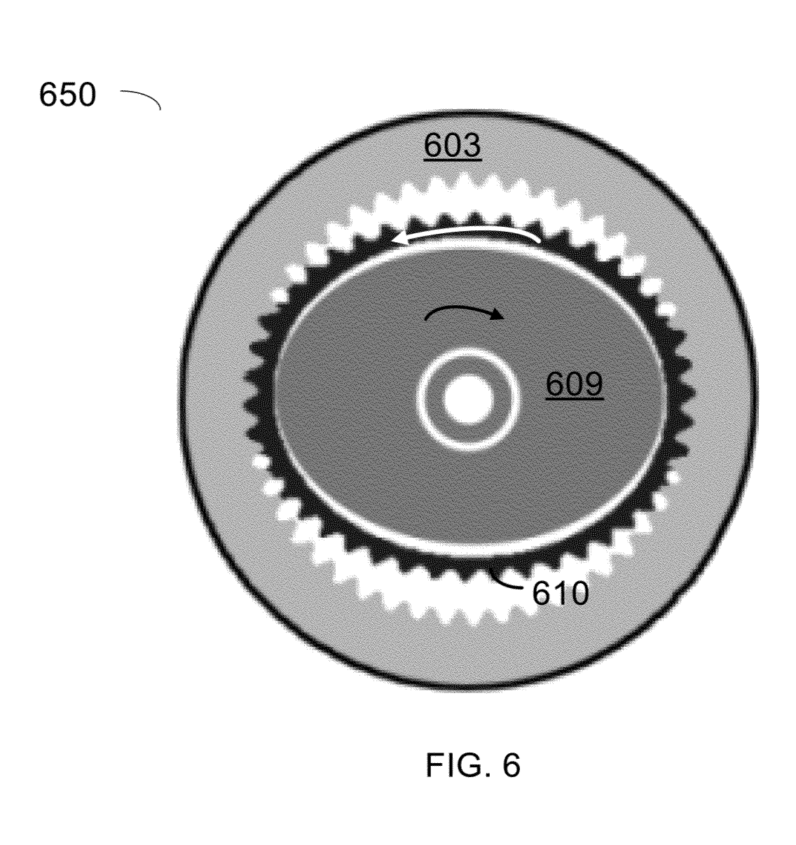

FIG. 6 illustrates a cross-sectional view of a coupling mechanism 650 for use in a rotary actuator according to embodiments disclosed herein. Coupling mechanism 650 may be a harmonic drive, according to some embodiments. A harmonic drive as shown in FIG. 6 may include a coupling input 609, an exterior gear 603, and an output gear 610. According to embodiments consistent with the present disclosure, output gear 610 may be formed of a flexible material. In some embodiments, coupling input 609 may be known as “wave generator,” and output gear 610 may be the soft end of a flex spline, firmly attached in the opposite side to an output shaft (out of the cross-sectional plane of FIG. 6). As coupling input 609 rotates about its axis, it produces a deformation of output gear 610. The deformation stretches gear 610 in the direction along the centers of two contact portions between gear 610 and coupling input 609. Gear 610 and coupling input 609 do not rotate in the same direction. As coupling input 609 rotates, the teeth on gear 610 that are meshed with the teeth in exterior gear 603 change. According to embodiments consistent with the present disclosure, the number of teeth in exterior gear 603, n₆₀₃, and the number of teeth in output gear 610, n₆₁₀, is different. In some embodiments, n₆₁₀<n₆₀₃. The difference between n₆₁₀ and n₆₀₃ induces a rotation in output gear 610 relative to exterior gear 603 as coupling input 609 presses different contact portions in gear 610. Thus, for every full rotation of coupling input 609, gear 610 rotates a slight amount backward relative to exterior gear 603. In some embodiments, coupling mechanism 650 including a harmonic drive provides low backlash and high torque capability compared to a planetary gear system such as in coupling mechanism 350.

In embodiments consistent with the present disclosure, coupling mechanism 650 may be included in a rotary actuator such as actuator 200. In such embodiments, coupling mechanism 650 may be as coupling mechanism 250. Thus, coupling input 609 may be as coupling input 209, output gear 610 may be coupled to output shaft 202, and exterior gear 603 may be fixed to frame 230. According to embodiments disclosed herein coupling input 609 rotates clockwise, output gear 610 rotates counter-clockwise, and exterior gear 603 is stationary relative to a rotary actuator frame. The rotation direction of each gear in coupling mechanism 650 is not limiting. In some embodiments consistent with the present disclosure the rotation of each gear in coupling mechanism 650 may follow the opposite direction, without loss of generality.

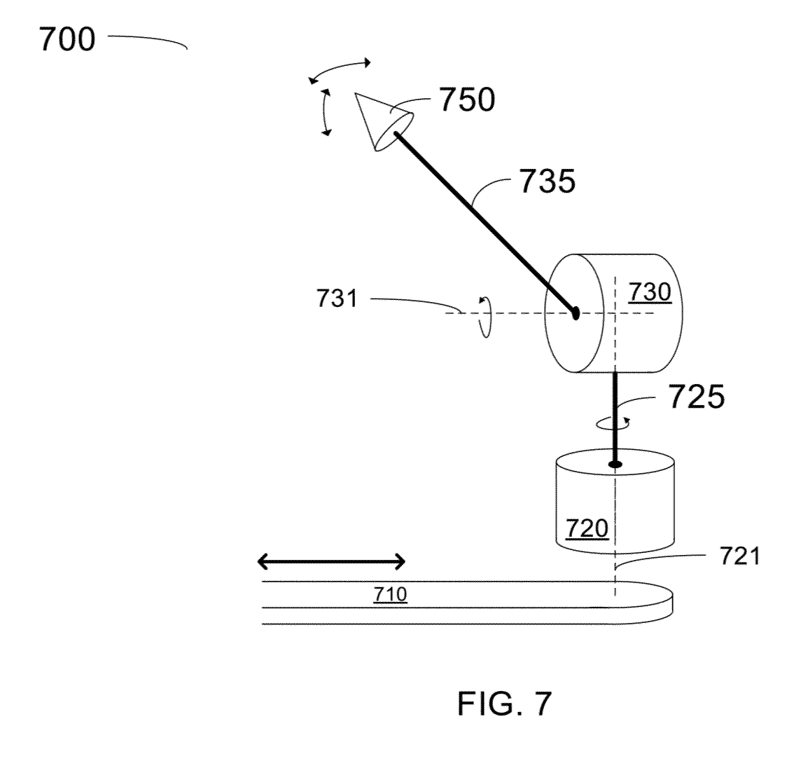

FIG. 7 illustrates a surgical manipulator 700 including rotary actuators 720 and 730, according to embodiments disclosed herein. Surgical manipulator 700 also includes a base or pedestal 710, a first arm 725 and a second arm 735. First arm 725 is coupled to an output shaft in rotary actuator 720, and provides support and rotation to rotary actuator 730 and second arm 735. Second arm 735 is coupled to rotary actuator 730, and may include a surgical instrument 750 in its distal end. Base 710 provides mechanical stability to surgical manipulator 700. In addition, some embodiments may include base 710 as a movable component, with the ability to perform linear motion as shown in FIG. 7.

According to some embodiments, the RA 721 of rotary actuator 720 may be oriented perpendicularly relative to the RA 731 of rotary actuator 730. In such configuration, rotary actuators 720 and 730 may provide pitch and yaw degrees of freedom for surgical instrument 750. In some embodiments RA 721 may be oriented vertically and RA 731 may be oriented horizontally (cf. FIG. 7). Some embodiments may have a configuration where RA 721 is oriented horizontally and RA 731 is oriented vertically. Some embodiments may include other orientations for RAs 721 and 731. Furthermore, some embodiments of surgical manipulator 700 consistent with the present disclosure may include more than two rotary actuators as disclosed herein and more than two arms. In some embodiments surgical manipulator may include a single rotary actuator as disclosed herein.

Embodiments of the invention described above are exemplary only. One skilled in the art may recognize various alternative embodiments from those specifically disclosed. Those alternative embodiments are also intended to be within the scope of this disclosure. As such, the invention is limited only by the following claims.

What is claimed is:

1. A rotary actuator for use in surgical instruments comprising:
a plurality of engine elements placed symmetrically around a rotation axis;
a disk in contact with the engine elements, centered on the rotation axis, the engine elements providing a first rotation to the disk;
an input shaft coupled to the disk to rotate with the first rotation; and
a coupling mechanism to provide a second rotation to an output shaft from the first rotation of the input shaft.

2. The rotary actuator of claim 1 wherein:
the first rotation is associated with a first torque and a first angular speed;
the second rotation is associated with a second torque and a second angular speed; and
the second torque is greater than the first torque.

3. The rotary actuator of claim 1 wherein the coupling mechanism comprises a planetary gear stage with a sun gear coupled to the input shaft and a plurality of planet gears coupled to the output shaft.

4. The rotary actuator of claim 3 wherein the planetary gear stage comprises an exterior gear attached to a rotary actuator frame.

5. The rotary actuator of claim 4 wherein:
the coupling mechanism comprises at least two gear stages sharing the exterior gear; and
a plurality of planet gears in one of the stages is coupled to the sun gear in the other stage.

6. The rotary actuator of claim 1 wherein the engine elements comprise piezo-electric elements having a longitudinal axis of operation.

7. The rotary actuator of claim 6 wherein the engine elements are arranged circumferentially about the rotation axis with their axis aligned with the rotation axis.

8. The rotary actuator of claim 6 wherein:
the engine elements are arranged circumferentially about the rotation axis with their axis in a plane perpendicular to the rotation axis.

9. The rotary actuator of claim 1 wherein the disk is a ceramic element supported by bearings.

10. The rotary actuator of claim 9 wherein the disk and the input shaft are a single component.

11. The rotary actuator of claim 1 wherein the coupling mechanism is contained within a rotary actuator frame.

12. The rotary actuator of claim 1 wherein an axis of the input shaft and an axis of the output shaft form an angle different from zero.

13. The rotary actuator of claim 1 wherein the coupling mechanism comprises a harmonic drive.

14. The rotary actuator of claim 1 comprising an encoder for detecting an angular position of at least one of the disk, the input shaft, or the output shaft.

15. The rotary actuator of claim 14 wherein the encoder comprises a sensor selected from a group consisting of a capacitance sensor, an electric sensor and an optical sensor.

16. The rotary actuator of claim 1 wherein the components are made of Magnetic Resonance Imaging (MRI) safe materials.

17. A surgical manipulator comprising:
a base to provide stability and support;
a first rotary actuator coupled to the base; and
a first arm coupled to the rotary actuator; wherein
the first rotary actuator comprises:
a plurality of engine elements placed symmetrically around a first rotation axis;
a disk in contact with the engine elements, centered on the rotation axis, the engine elements providing a first rotation to the disk;
an input shaft coupled to the disk to rotate with the first rotation; and
a coupling mechanism to provide a second rotation to an output shaft from the first rotation of the input shaft.

18. The surgical manipulator of claim 17 further comprising:
a second rotary actuator coupled to the first arm, having a second rotation axis;
a second arm coupled to the second rotary actuator;
a surgical instrument coupled to the second arm.

19. The surgical manipulator of claim 18 wherein the first rotation axis and the second rotation axis are perpendicular to each other.

20. The surgical manipulator of claim 17 wherein the base is adapted to move in a linear direction.
